(19) United States
(12) Patent Application Publication
(10) Pub. No.: US 2006/0100352 A1
(43) Pub. Date: May 11, 2006

(54) Vinyl amide-containing adhesive compositions for plastic bonding, and methods and products utilizing same

(76) Inventors: John R. Arnold, Burlington, CT (US); Vu V. Lam, Torrington, CT (US); Maria Fe Aton Audia, Torington, CT (US)

Correspondence Address: Ira S. Dorman, Suite 200, 330 Roberts Street, East Hartford, CT, 06108, US

(21) Appl. No.: 10/984,428

(22) Filed: Nov. 9, 2004

(51) Int. Cl. A61K 9/16

(52) U.S. Cl. 524/543

(57) ABSTRACT

An adhesive composition for bonding PET substrates, particularly components of clamshell packaging, includes a polymerizable vinyl amide compound, a comonomer, an oligomer, and a photoinitiator.

### BACKGROUND OF THE INVENTION

The need has long existed for an adhesive composition that is capable of rapidly producing an effective, transparent, and otherwise aesthetic bond with polyethylene terephthalate resins (PET, RPET and PET-G, collectively referred to hereinafter as “PET resins”) and other plastics. So-called “clamshell” packaging is currently in widespread use for myriad different products, and offers a number of benefits, including tamper and pilferage resistance, aesthetic appeal, product visibility and effective display, etc. A clamshell package is usually comprised of two semi-rigid components made from a clear plastic material (typically polyvinyl chloride) and constructed to engage one another so as to enclose the contained product or products in a cavity molded into one or both of the components. While the two components of the package may be formed either separately or as a single piece, joined by an integral hinge, in their closed condition the components will usually be tenaciously secured to one another and sealed peripherally, so as to provide tamper resistance and to otherwise serve their intended purposes. Securement and sealing of the components has heretofore been effected by plastic welding, using heated elements or ultrasonic or radio frequency techniques, or by adhesive bonding.

It would be highly desirable, for a number of reasons, to employ PET resins for clamshell packaging in place of, or preferably as an optional alternative to, the PVC resins that are now widely utilized. In particular, the potential for PVC (and PVDC and related halogen-containing plastics) to degrade to environmentally inimical chemical species strongly disfavors the use of those resins. The inability to successfully fuse and weld elements of PET resins, however, presents a serious impediment to such substitution or alternative use (PET resins can of course be modified to enable welding, but the required modifications add significantly to the cost of the material).

As a practical matter, therefore, the feasibility of using PET resins for fabricating clamshell packaging would depend upon the availability of an adhesive composition that is capable of rapidly (i.e., in no longer than about five seconds, and preferably in from about 0.5 to one or two seconds) producing strong and tenacious bonds with PET substrates and elements, which composition would most desirably satisfy other criteria as well (e.g., low vapor pressure, non-toxicity, photoinitiated curing to a clear state, relatively low cost, recyclability, etc.). As far as is known, no such adhesive composition has heretofore been developed or made available.

Except in instances in which the sealed, product-containing package is produced by a continuous, in-line procedure (i.e., using a so-called “form, fill and seal” technique), the fabricated clamshell units must be removed from the molding equipment and stored, normally as nested stacks, until they are needed. This however gives rise to problems of separation of the individual pieces from one another. Mold-release, or slip agents (typically, silicone liquids and stearate products), are usually applied to one surface of each piece to facilitate its removal from the stack; but the production of an adequate adhesive bond through such a substance has been problematic. To be satisfactory, therefore, it is important that a PET adhesive composition possess the additional property of effective penetration through mold-release agents, and like abherents.

The prior art appears not to have addressed the foregoing. For example, Goldberg et al. U.S. Pat. No. 5,094,876 teaches a method for modifying plastic surfaces of articles (which are specifically adapted for contacting living tissues), by forming thereon a hydrophilic graft polymer coating comprised of n-vinyl pyrrolidone (NVP), hydroxyethyl methacrylate (HEMA), or a mixture thereof, using gamma or electron beam irradiation. The patent appears to provide no suggestion for use of any such copolymer as or in an adhesive for a PET substrate.

Adhesive bonding of PET structures is discussed in Ewing U.S. Pat. No. 5,236,749, which is directed to a blister packaging system wherein the front blister is made from PET (or PET-G or R-PET) and may be adhesively bonded to a back film or sheet made from polyethylene. No specific adhesive composition is disclosed.

Similarly, Burns et al. U.S. Pat. No. 5,486,390 provides blister packaging wherein a first sheet of PET, PVC, or PVC-coated PVDC is adhered releasably to a sheet of OPP (i.e., tamper resistance is neither contemplated nor afforded). The patent itself does not disclose specific adhesives, but instead references the acrylic-based terpolymer adhesives disclosed in U.S. Pat. No. 3,753,769 and the PVDC adhesives disclosed in U.S. Pat. No. 4,447,494; no composition pertinent to the present invention appears to be taught in either patent.

Miller et al. U.S. Pat. No. 6,592,978 provides a three-part laminated material for use in making blister packages, wherein the central core of the laminate is preferably a fluoropolymer-based sheet material, but may alternatively be PVDC. Adhesive bonding is effected using a two-component, water-based polyurethane, dry-bond laminating adhesive, cured by epoxy-amine cross-linking chemistry.

Appelbaum et al. U.S. Pat. No. 5,259,169 disclosures the use of LIGHT WELD adhesives for bonding of clamshell pieces fabricated from PVC. The LIGHT WELD adhesives are acrylate-based compositions available from Dymax Corporation, of Torrington, Conn., to which corporation the instant application is assigned. In U.S. Pat. No. 6,619,496, Appelbaum provides clamshell-type packages molded from a “suitable clear plastic material” and having a rib that can be secured to a package wall using a UV-curable adhesive; no adhesive composition appears to be disclosed.

### SUMMARY OF THE INVENTION

Accordingly, it is a broad object of the present invention to provide an adhesive composition for effective bonding of PET resin substrates and elements.

A more specific object of the invention is to provide such an adhesive composition which is capable of producing an effective bond to PET resin substrates and elements through surface contamination, such as in particular coatings of silicone liquids and other mold-release agents, and the like, as well as for producing effecting bonds to other synthetic resinous substrates and elements, especially of PVC and PVDC.

Another specific object of the invention is to provide such an adhesive composition in which curing is rapid and is initiated by actinic radiation, especially ultraviolet and/or visible radiation.

A related specific object of the invention is to provide such an adhesive composition which facilitates automated bonding of PET resin elements, especially for the securement and sealing of clamshell packaging components fabricated from a PET resin, but also for producing laminates and various other assemblies.

Additional objects of the invention are to provide a method for the production of clamshell packages comprised of components fabricated from a plastic that is environmentally benign, such as in particular PET resins, and to provide packages produced by such a method.

It has now been found that certain of the foregoing and related objects of the invention are attained by the provision of photocurable adhesive composition comprised of the following ingredients, in amounts by weight of the composition: at least about five percent of at least one polymerizable vinyl amide compound; at least about five percent of a monomer copolymerizable with the vinyl amide compound; at least about fifteen percent of an oligomer reactive with the vinyl amide compound and the copoloymerizable monomer; and about 0.5 to ten percent of a photoinitiator for effecting curing of the ingredients to a solid adhesive mass. The vinyl amide compound employed is either a vinyl amide having a ring structure containing a conjugated nitrogen atom, or a vinyl amide having the structural formula: 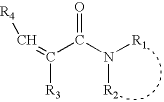
wherein R₁ represents —H or, in combination with R₂, an aliphatic or ether ring fragment attached to the nitrogen atom in the molecule depicted; R₂ represents —H, or one of the organic groups consisting of aliphatic, alcohol, ketone, aldehyde, ether, amide, (meth)acrylate, acrylamide, and aromatic, or, in combination with R₁, the ring fragment so attached; R₃ represents —H or —CH₃; and R₄ represents —H or —CH═O. The organic groups represented by R₂ in the foregoing structural formula may, more particularly, be —CH₂OH, —CH(CH₃)₂, —C(CH₃)₂CH₂—C(═O)CH₃, —CH₂OCH₂CH(CH₃)₂, —CH₂—NH—C(═O)—CH═CH₂, or —CH₂CH₂—CH₂—N(CH₃)₂, and the ring fragment may, more particularly, be —CH₂CH₂OCH₂CH₂—. Specific nitrogen ring-containing vinyl amide compounds that are suitable for use in the instant adhesive compositions include acrylamide, methacrylamide, n-(hydroxy-methyl)acrylamide, N-isopropyl acrylamide, diacetone acrylamide, N-(iso-butoxymethyl)acrylamide, N,N′-methylenebisacrylamide, N-3-dimethyl-aminopropylmethacrylamide, 4-acrylolmorpholine, and maleamic acid. Suitable vinyl amides having the structural formula given above include N-vinyl acetamide, N-vinylcarbazole, N-vinylcaprolactam, N-vinylimiadzole, 1-vinyl 2-pyrrolidinone, N-vinylphthalimide, and 4-vinyl pyridine.

The adhesive composition of the invention may also contain (in addition to other conventional ingredients discussed below) about 0.5 to 20 percent (based upon the weight of the composition) of a filler resin and/or about 0.5 to 10 percent of a thickener. As a practical matter, there appears to be no upper limit upon the amounts of the vinyl amide compound, the copolymerizable monomer, and the oligomer employed.

Other objects of the invention are attained by the provision of a method of bonding a first substrate to a second substrate utilizing the photocurable adhesive composition herein described. In accordance with the method, the adhesive composition is applied to at least one surface of at least one of the substrates; the substrates are brought together with the adhesive composition therebetween; and the adhesive composition is exposed to actinic radiation to which the photoinitiator is responsive for effecting curing of the adhesive composition.

In specific embodiments of the method, either or both of the substrates is comprised of a polyethylene terephthalate resin; at least one of the substrates is substantially transparent to the actinic radiation, with the step of irradiation being effected subsequent to the step of bringing the substrates together; and/or the first and second substrates are components of a clamshell package. The components may desirably be constructed for mechanical interengagement and so interengaged at the time the adhesive composition is exposed to the actinic radiation, and at least one surface of at least one substrate may have an abherent coating thereon, typically comprising a silicone substance or a fatty acid reaction product, upon which the adhesive composition is applied.

Additional objects of the invention are attained by the provision of a clamshell package comprised of a pair of components having elements fabricated from at least one synthetic resinous material and bonded to one another to secure the components together for maintaining the package in closed condition, the elements being bonded by a cured, interposed adhesive having the composition herein set forth. In preferred embodiments one or both of the packaging components, and the bonded element thereof, will be comprised of a polyethylene terephthalate resin that is substantially transparent to the actinic radiation employed for effecting curing, and an abherent coating may be present beneath the interposed adhesive composition.

### BRIEF DESCRIPTION OF THE DRAWINGS

FIG. 1 of the drawings is an exploded perspective view of a clamshell package embodying the present invention;

FIG. 2 is a perspective view of the package of FIG. 1 with the components assembled and adhesively bonded to one another; and

FIG. 3 is an exploded, fragmentary perspective view of one form of elements that can be employed for mechanically interengaging the packaging components, drawn to a scale greatly enlarged from that of FIGS. 1 and 2.

### DETAILED DESCRIPTION OF THE ILLUSTRATED EMBODIMENTS

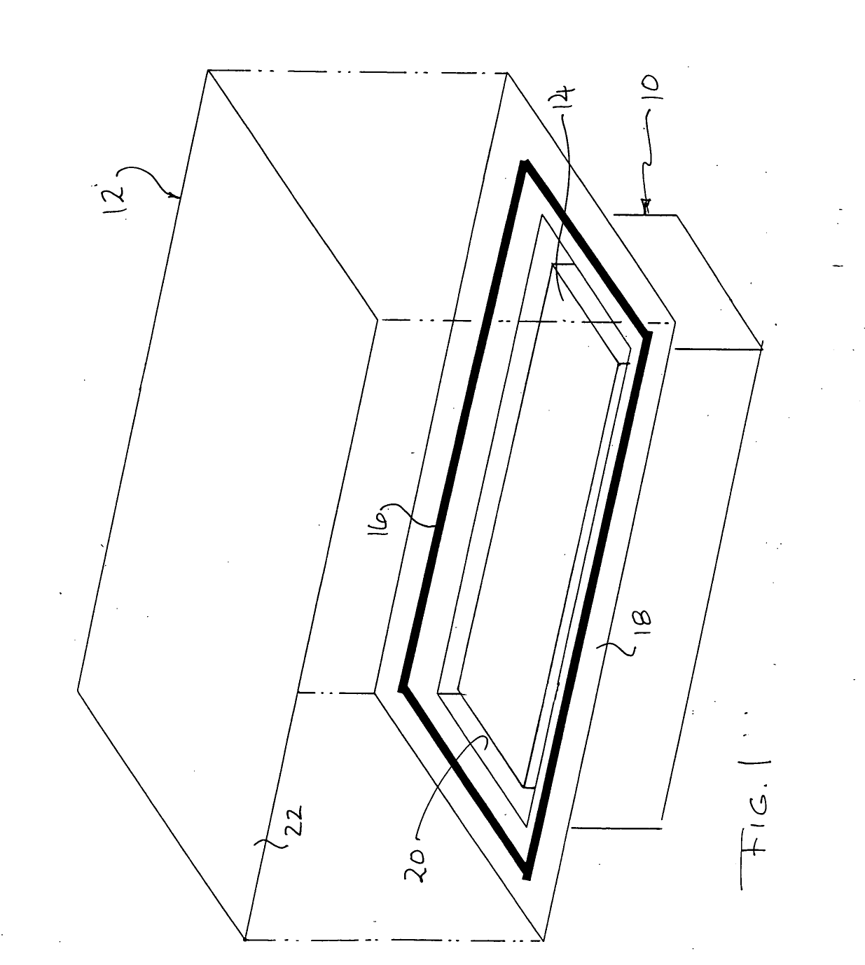

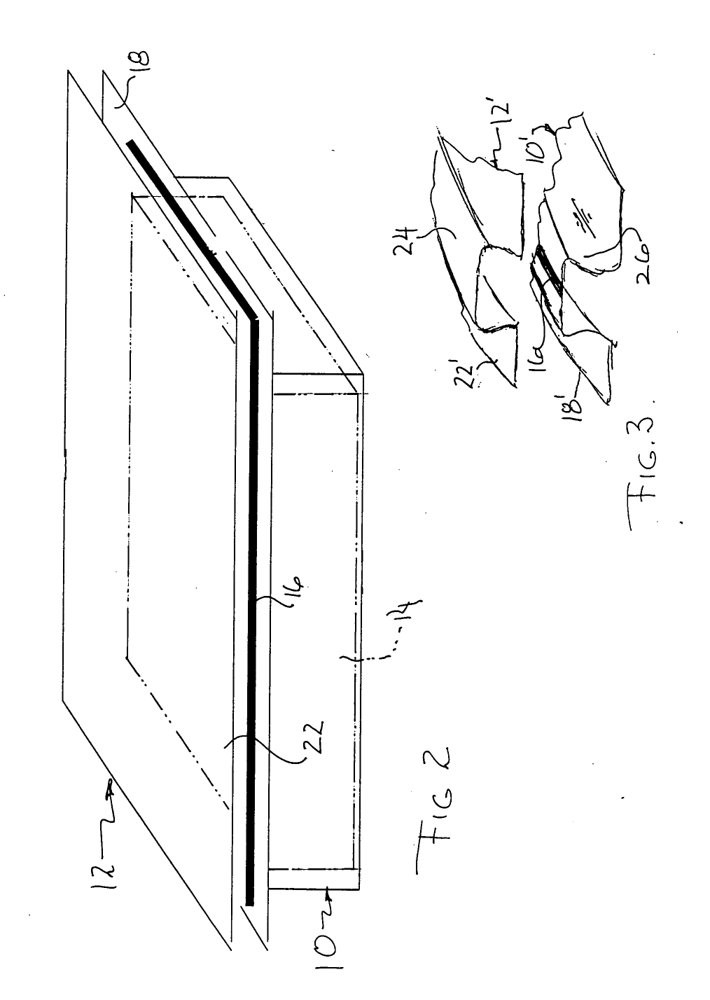

FIGS. 1 and 2 show a base, or container, component and a lid, or cover, component of clamshell packaging, generally designated by the numerals 10 and 12 respectively and molded from a PET resin that is transparent to UV radiation. A line of a UV-curable adhesive composition 16, embodying the present invention, is applied to the flange 18 on the base component 10 surrounding the cavity 20 formed therein, in which is contained a product 14.

As seen in FIG. 2, the deposit 16 is interposed between the flange 18 and a marginal portion 22 of the lid component 12. After curing, the adhesive serves to secure the components 10, 12 to one another and to produce a seal therebetween, thus securely enclosing the product 14.

A coating of a silicone mold-release agent (not visible), present on at least one of the facing surfaces of the components 10, 12, underlies the adhesive deposit 16 and is penetrated thereby to produce, upon curing, a tenacious bond between the PET elements. As seen in FIG. 3, the marginal portion 22′ of a lid 12′ and the flange 18′ on a base component 10′ are formed with mating clamping channel elements 24, 26, which interengage in a snap-fit relationship to assist in maintaining the flange 18′ and marginal lid portion 22′ in position while the package component are brought into position for curing (e.g., in an automated system). Needless to say, other forms of interengaging elements (e.g., clamping buttons) can be employed if preferred.

Illustrative of the efficacy of the present invention are the following specific examples:

### EXAMPLES

Various vinyl amides were tested for use in adhesive compositions for bonding to PET substrates. The formulations were prepared by admixing, with each of the vinyl amides listed in Table Two (referred to as a “Test chemical”), the ingredients listed in Table One, in the amounts set forth. The vinyl amide was used either alone (Test A) or, in instances in which it exhibited poor solubility in the mixture, in combination with acryloyl morpholine (Test B) or N,N-dimethylamide (Test C).

TABLE ONE

| Ingredients | Test A | Test B | Test C |
|---|---|---|---|
| Test Chemical | 24.00 | 5.00 | 2.00 |
| 4-acryloylmorpholine | — | 23.50 | — |
| N,N-dimethyl acrylamide | — | — | 24.00 |
| alkoxylated acrylate | 24.00 | 24.00 | 24.00 |
| Isobornyl acrylate, IBOA | 10.00 | 10.00 | 10.00 |
| Oligomer | 39.50 | 35.00 | 37.50 |
| Photoinitiator (s) | 2.50 | 2.50 | 2.50 |
| | 100.00 | 100.00 | 100.00 |

TABLE TWO

| | | | PET | | | |
|---|---|---|---|---|---|---|
| | | | adhesion | | | |
| | Test | | Relative | Quality | Speed | |
| Test Chemical | Formula | Solubility | to IBOA | of Cure | of Cure | Rank 1 = best |
| 4-acryloylmorpholine | A | Soluble | 9 | Good | Very Fast | 1 |
| N-Vinylpyrrolidone | A | Soluble | 8 | Good | Fast | 2 |
| N,N-dimethyl acrylamide | A | Soluble | 7 | Good | Fast | 3 |
| N-isopropyl acrylamide | B | Soluble | 5 | Good | Fast | 4 |
| N-Vinylimidazole | A | Soluble | 5 | Good | Slow | 4 |
| 4-Vinyl pyridine | A | Soluble | 5 | Good | Average | 4 |
| 9-Vinylcarbazole | A | Soluble | 5 | Good | Average | 4 |
| Acrylamido methyl cellulose acetate | A | Soluble | 5 | Good | Fast | 5 |
| Maleamic acid | C | Solubility, | 5 | Good | Fast | 5 |
| | | falls out next | | | | |
| Diacetone acrylamide | A | Soluble | 5 | Good | Fast | 5 |
| Methacrylamide | A | Soluble | 5 | Good | Fast | 5 |
| N-Vinyl pthalimide | A | Soluble | 4 | Good | Average | 5 |
| N-Vinylcaprolactam | A | Soluble | 4 | Good | Fast | 5 |
| N-(isobutoxymethyl)acrylamide | A | Soluble | 3 | Good | Average | 6 |
| N-3-Dimethylaminopropyl | B | Soluble | 3 | Good | Slow | 6 |
| methacrylamide | | | | | | |
| Isobornyl acrylate | A | Soluble | 2 = control | Good | Average | 7 |
| 2-Acrylamido-2-methyl | C | Very poor | 1 | poor | Fast | 8 |
| propanesulfonic Acid | | | | | | |
| n-Vinyl-n-methyl acetamide | A | Soluble | 1 | poor | Slow | 8 |
| N,N′-Methylenebisacrylamide | A, B, or C | Insoluble | not tested | not tested | not tested | not tested |
| n-(Hydroxymethyl)acrylamide | A, B, or C | Insoluble | not tested | cloudy | not tested | not tested |
| Key to adhesion (scale 1-10) | | | | | | |

| | |
|---|---|
| 1 | poor, unzips with less pressure (adhesion) than isoborynl acrylate control formula |
| 2 | Isobornyl acrylate control formula unzips with very little pressure |
| 3 | slightly better than control, need the same strength as pulling transparent tape, -1-2 psi in peel |
| 4 | better than 3 |
| 5 | much better than 4 |
| 7 | same as 8 but with less pressure |
| 8 | tabs can only slowly be pulled away |
| 9 | adhesion such that the tabs cannot be pulled away from each other. |
| 10 | must tear the plastic to break |

In each test the composition was applied between two strips of UV-transparent PET resin, measuring 4 inches by 0.75 inch by 0.031 inch, leaving an end portion of each strip free of adhesive to provide an adjacent pair of gripping tabs. Only one of the confronting strip surfaces had been coated with a silicone mold-release agent. The strips were pressed together, with the test adhesive interposed, and the assembly was exposed, for a period of about 30 seconds, to a dose of UV radiation sufficient to ensure complete curing of each formulation.

Adhesion was evaluated by a manual “T-peel” procedure, in which the unbonded tabs on the strips were pulled away from one another, in opposite directions. Table Two specifies the test formula in which each vinyl amide test chemical was employed, the level of solubility of the test chemical, a subjective evaluation of its level of adhesion (1=poor, 10=good) relative to a formulation based upon IBOA, the relative quality and speed of cure, and a ranking designation; an adhesion scale key is also set forth.

The poor quality of cure of the formulation containing 2-acrylamido-2-methyl propanesulfonic acid is believed to be attributable to the strongly acidic nature of the sulfonic acid group present in the molecule. The poor quality of cure of the formulation containing n-vinyl-n-methyl acetamide is attributed to poor UV-initiation response, and the two acrylamides listed last were insufficiently soluble to warrant testing.

Without being bound to or limited by any theory of operation, it is believed that the vinyl amides employed in the instant compositions serve to penetrate the surface of PET elements, and contaminates thereon, so as to thereby promote and ensure strong bonding. Moreover, they do so without undue attack upon the PET resin (e.g., etching), such as would produce undesirable cloudiness, visibility, crazing, and the like.

A wide range of compounds may be employed as comonomers in combination with the vinyl amides specified, and the use of specific compounds does not appear to be critical. Suitable reactive acrylate monomers include monofunctional, difunctional, and polyfunctional acrylates and methacrylates, albeit monofunctional compounds, which cure to elastic homopolymers, are generally preferred. The acrylate comonomers will usually be reaction products of acrylic acid and/or methacrylic acid with one or more mono-, di- or poly-basic, substituted or unsubstituted, alkyl (C₁ to C₁₈), aryl or aralkyl alcohols, and acrylates in which the alcohol moiety contains a polar substituent (e.g., an hydroxyl, amine, halogen, cyano, heterocyclic or cyclohexyl group) may beneficially promote crosslinking or other intermolecular bonding.

Albeit suitable such monomers and prepolymers are well known in the art (see for example U.S. Pat. Nos. 4,429,088 and 4,451,523), the following acrylates and corresponding methacrylates, used alone or in combination with one another, might be identified: hydroxyethyl(meth)acrylate, hydroxyproply(meth)acrylate, ethylhexyl(meth)acrylate isobornyl acrylate, tetrahydrofurfuryl acrylate, diethyleneglycol diacrylate, 1,4-butanediol diacrylate, butylene glycol diacrylate, neopentyl glycol diacrylate, octylacrylate and decylacrylate (normally in admixture), polyethyleneglycol diacrylate, trimethylcyclohexyl acrylate, benzyl acrylate, butyleneglycol diacrylate, polybutyleneglycol diacrylate, tripropyleneglycol diacrylate, trimethylolpropane triacrylate, di-trimethylolpropane tetraacrylate, pentaerythritol tetraacrylate, phenyl glycidyl ether acrylate, neodecanoate vinyl ester, ethoxylated phenoxy ethyl acrylate, and di-pentaerythritol pentaacrylate. The properties imparted to the adhesive composition will generally vary in proportion to the amount used and number of acrylate groups present in the molecule, and optimal concentrations will consequently be selected accordingly.

The use of particular oligomers (which desirably afford cross linking, and add toughness to the cured adhesive) also does not appear to be critical to the successful functioning of the present adhesives. Free-radical reactive oligomers will normally be employed, alone or, where appropriate, in combination with cationic-reactive oligomers. Oligomers suitable for use are also well known in the art, and comprise vinyl polymers, acrylic polymers, polyester elastomers, glycol polymers, acrylated epoxies, natural and synthetic rubbers, polyester acrylates, epoxy acrylates, polyether acrylates, alkyd acrylates, polyol acrylates, and the like. The use of the urethane polymers and prepolymers will however often be found most beneficial, with the latter being especially desirable due to the potential that they afford for further reaction of their pendant isocyanate groups with a reactive functionality provided by the vinyl amide and comonomer ingredients. Disocyanate-capped polyethers and polyesters, acrylated by reaction with hydroxyethyl acrylate or hydroxyethyl methacrylate and having a molecular weight of about 400 to 6,000, are particularly preferred, and suitable such products are available from Bomar Specialties Company, of Winsted, Conn.

Although the concepts of the invention are also not dependent upon the use of any particular free radical photoinitiator, those that respond in the ultraviolet and/or visible spectral regions will normally be preferred, as a practical matter. Indeed, photoinitiators that respond to radiation that includes visible wavelengths will often be employed to greatest advantage. While suitable photoinitiators will be apparent to those skilled in the art, specific illustrative compounds that might be identified are dimethoxy-2-phenylaceto-phenone (IRGACURE 651), 1-hydroxycyclohexylphenyl ketone (IRGACURE 184), and 2-hydroxy-2-methyl-1-phenylpropane-1-one (DAROCUR 1173). Other conventional free radical photoinitiators that might be utilized herein include hexyltriaryl borates, camphorquinone, 2-benzyl-2-N, N-dimethyl amino-1-(4-morpholinophenyl)-1-butanone (IRGACURE 369); bis (μ⁵-2,4-cycloypentadien-1-yl) bis [2,6-difluoro-3-(1 H-pyrrol-1-yl) phenyl] titanium (IRGACURE 784DC); DAROCUR 4265, which is a 50 percent solution of 2,4,5-trimethyl benzoyl diphenyl-phosphine oxide in DAROCUR 1173; and IRGACURE 819, phosphine oxide, phenyl-bis(2,4,6-trimethyl) benzoyl (all of the foregoing photoinitiators that are identified by trademarks are available from Ciba Specialty Chemicals, of Tarrytown, N.Y.). A further listing of suitable photoinitiators may be obtained by reference to U.S. Pat. No. 4,820,744, particularly at line 43, column 4 through line 7, column 7 (which disclosure is incorporated hereinto by reference thereto). Cationic photoinitiators may also be employed, to provide a further cure mechanism in appropriate circumstances.

In many instances it will be highly desirable to incorporate into the adhesive composition a resinous filler, which can serve to promote adhesion, to minimize shrinkage, to add toughness, to accelerate cure, and to provide other benefits. The resinous fillers employed in the present compositions will usually be acrylic resins (such as the carboxy functional ethyl methacrylate product available from Dia America, Inc. under the trademark DIANAL Pb-204, and the methyl/n-butyl methacrylate copolymer product available from Lucite International, Inc., under the trademark ELVACITE 2550) which have (surprisingly) been found to produce cross-linking, particularly when exposed to UV radiation. However, cellulosic resins (such as ethyl cellulose, hydroxyethyl cellulose, and cellulose acetate butyrate), and epoxy resins, might also be employed advantageously, in appropriate circumstances.

About 0.1 to 10 weight percent of acrylic acid or methacrylic acid may beneficially be employed in the instant compositions to increase adhesion. A tautomeric acid constituent may also contribute to bond strength; although maleic acid is preferred, other acids capable of cyclic tautomerism can be used, employed, as well, such as malic, salicylic, itaconic and phthalic. Other materials that may be incorporated into the present adhesives include, for example, “inert” fillers such as wood flour, cornstarch, glass fibers, cotton linters, mica, alumina, silica, and the like, used to modify viscosity (thixotropes, thickeners, viscosity reducers), improve impact resistance, and for other purposes, and it is conventional to include small percentages of silane coupling agents to increase moisture resistance as well as to enhance bond strength. Substances such as dyes, flame retarders, stabilizers (e.g., the quinones and hydroquinones), plasticizers, antioxidants, and the like, may of course be incorporated as well.

Thus, it can be seen that the present invention provides an adhesive composition for effective bonding of PET resin substrates and elements and, more specifically, an adhesive composition that is capable of producing an effective bond to PET resin substrates through surface contamination (e.g., silicone liquids and other mold-release agents, and the like), as well as to substrates of other synthetic resinous materials, especially PVC and PVDC. It will be appreciated that, as used herein, the term “bonding” is intended to have broad connotation, and to include adhesion to substrates and elements in a wide range of applications, such as for producing laminates, devices, assemblies, etc. Curing of the composition is rapid (i.e., it normally occurs in five seconds or less, and often in a period of 0.5 to one or two seconds) and can readily be initiated by actinic radiation if formulated to do so, and the composition facilitates automated bonding of PET resin elements, especially for the securement and sealing of clamshell packaging components fabricated from a PET resin. The invention additionally provides a method for the production of clamshell packaging fabricated from plastics that are environmentally benign, such as in particular PET resin components, as well as packages produced by such a method.

Having thus described the invention, what is claimed is:

1. A photocurable adhesive composition, adherent to polyethylene terephthalate resin substrates, comprised of the following ingredients, in amounts by weight of said composition: at least about five percent of at least one polymerizable vinyl amide compound; at least about five percent of a monomer copolymerizable with said vinyl amide compound; at least about fifteen percent of an oligomer reactive with said vinyl amide compound and said copoloymerizable monomer; and about 0.5 to ten percent of a photoinitiator for effecting curing of said ingredients to a solid adhesive mass; said at least one vinyl amide compound being selected from a first group consisting of vinyl amides having ring structures containing a conjugated nitrogen atom, and vinyl amides having a first structural formula: 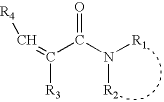
wherein R₁ represents —H or, in combination with R₂, an aliphatic or ether ring fragment attached to the nitrogen atom in said first structural formula; R₂ represents —H, or one of the organic groups aliphatic, alcohol, ketone, aldehyde, ether, amide, (meth)acrylate, acrylamide, or aromatic, or, in combination with R₁, said aliphatic or ether ring fragment so attached; R₃ represents —H or —CH₃; and R₄ represents —H or —CH═O.

2. The adhesive composition of claim 1 wherein said one organic group, represented by R₂, is —CH₂OH, —CH(CH₃)₂, —C(CH₃)₂CH₂C(═O)CH₃, —CH₂OCH₂CH(CH₃)₂, —CH₂—NH—C(═O)—CH═CH₂, or —CH₂CH₂CH₂—N(CH₃)₂, and wherein said ring fragment is —CH₂CH₂OCH₂CH₂.

3. The adhesive composition of claim 1 wherein said vinyl amide compound has a ring structure containing a nitrogen atom, and is selected from a second group consisting of acrylamide, methacrylamide, n-(hydroxy-methyl)acrylamide, N-isopropyl acrylamide, diacetone acrylamide, N-(iso-butoxymethyl)acrylamide, N,N′-methylenebisacrylamide, N-3-dimethyl-aminopropylmethacrylamide, 4-acrylolmorpholine, and maleamic acid.

4. The adhesive composition of claim 1 wherein said vinyl amide has said first structural formula, and is selected from a third group consisting of N-vinylacetamide, N-vinylcarbazole, N-vinylcaprolactam, N-vinylimiadzole, 1-vinyl 2-pyrrolidinone, N-vinylphthalimide, and vinyl pyridine.

5. The adhesive composition of claim 1 additionally including from about 0.5 to 20 percent of a filler resin, based upon the weight of said composition.

6. The adhesive composition of claim 1 additionally including from about 0.5 to 10 percent of a thickener, based upon the weight of said composition.

7. A method of bonding a first substrate to a second substrate, comprising of the steps: providing a first substrate and a second substrate; providing a photocurable adhesive composition of the following ingredients, in amounts by weight of said composition: at least about five percent of at least one polymerizable vinyl amide compound, at least about five percent of a monomer copolymerizable with said vinyl amide compound, at least about fifteen percent of an oligomer reactive with said vinyl amide compound and said copoloymerizable monomer, and about 0.5 to ten percent of a photoinitiator for effecting curing of said ingredients to a solid adhesive mass; applying said adhesive composition to at least one surface of at least one of said first and second substrates; bringing said first and second substrates together with said adhesive composition therebetween; and exposing said adhesive composition to actinic radiation to which said photoinitiator is responsive for effecting curing of said adhesive composition; said at least one vinyl amide compound being selected from a first group consisting of vinyl amides having ring structures containing a conjugated nitrogen atom, and vinyl amides having a first structural formula: 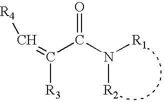
wherein R₁ represents —H or, in combination with R₂, an aliphatic or ether fragment attached to the nitrogen atom in said first structural formula; R₂ represents —H, or one of the organic groups aliphatic, alcohol, ketone, aldehyde, ether, amide, (meth)acrylate, acrylamide, or aromatic, or, in combination with R₁, said aliphatic or ether ring fragment so attached; R₃ represents —H or —CH₃; and R₄ represents —H or —CH═O.

8. The method of claim 7 wherein said one organic group, represented by R₂, is —CH₂OH, —CH(CH₃)₂, —C(CH₃)₂CH₂—C(═O)CH₃, —CH₂OCH₂CH(CH₃)₂, —CH₂—NH—C(═O)—CH═CH₂, or —CH₂CH₂—CH₂—N(CH₃)₂, and wherein said ring fragment is —CH₂CH₂OCH₂CH_{2—}.

9. The method of claim 7 wherein at least said first substrate is comprised of a polyethylene terephthalate resin.

10. The method of claim 7 wherein at least one of said first and second substrates is substantially transparent to said actinic radiation, and wherein said step of exposing said adhesive composition to said actinic radiation is effected subsequent to said step of bringing said substrates together.

11. The method of claim 7 wherein said second substrates is also comprised of a polyethylene terephthalate resin.

12. The method of claim 10 wherein said first and second substrates are components of a clamshell package.

13. The method of claim 12 wherein said components of said clamshell package are constructed for mechanical interengagement, and wherein said step of exposing said adhesive composition to said actinic radiation is effected with said components mechanically interengaged.

14. The method of claim 7 wherein said at least one surface of said at least one substrate has mold-release agent thereon, upon which mold-release agent said adhesive composition is applied.

15. The method of claim 14 wherein said mold-release agent comprises a silicone substance.

16. A clamshell package comprised of a pair of components having elements fabricated from at least one synthetic resinous material and bonded to one another to secure said components together for maintaining said package in closed condition, said elements of said components being bonded by a cured, interposed adhesive composition comprised of the following ingredients, in amounts by weight of said composition: at least about five percent of at least one polymerizable vinyl amide compound; at least about five percent of a monomer copolymerizable with said vinyl amide compound; at least about fifteen percent of an oligomer reactive with said vinyl amide compound and said copoloymerizable monomer; and about 0.5 to ten percent of a photoinitiator for effecting curing of said ingredients to a solid adhesive mass; said at least one vinyl amide compound being selected from a first group consisting of vinyl amides having ring structures containing a conjugated nitrogen atom, and vinyl amides having a first structural formula: 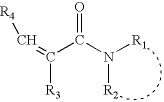
wherein R₁ represents —H or, in combination with R₂, an aliphatic or ether ring fragment attached to the nitrogen atom in said first structural formula; R₂ represents —H, or one of the organic groups aliphatic, alcohol, ketone, aldehyde, ether, amide, (meth)acrylate, acrylamide, or aromatic, or, in combination with R₁, said aliphatic or ether ring fragment so attached; R₃ represents —H or —CH₃; and R₄ represents —H or —CH═O.

17. The package of claim 16 wherein said one organic group, represented by R₂, is —CH₂OH, —CH(CH₃)₂, —C(CH₃)₂CH₂—C(═O)CH₃, —CH₂OCH₂CH(CH₃)₂, —CH₂—NH—C(═O)—CH═CH₂, or —CH₂CH₂—CH₂—N(CH₃)₂, and wherein said ring fragment is —CH₂CH₂OCH₂CH_{2—}.

18. The package of clam 17 wherein at least one of said components, and said bonded element thereof, is comprised of a polyethylene terephthalate resin.

19. The package of claim 17 wherein said bonded element of at least one of said components is substantially transparent to actinic radiation to which said photoinitiator in said adhesive composition is responsive for effecting curing thereof.

20. The package of claim 17 wherein said bonded element of at least one of said components has a mold-release agent thereon underlying said interposed adhesive composition.

21. The method of claim 20 wherein said mold-release agent comprises a silicone substance.
